(19) United States
(12) Patent Application Publication
(10) Pub. No.: US 2004/0038329 A1
(43) Pub. Date: Feb. 26, 2004

(54) Methods for monitoring treatment of helicobacter infection and for predicting the likelihood of successful eradication

(76) Inventors: Robert Llewellyn Clancy, New South Wales (AU); Thomas Julius Borody, New South Wales (AU); Gerarld Pang, New South Wales (AU); Zhigang Ren, New South Wales (AU)

Correspondence Address: FISH & RICHARDSON PC, 225 FRANKLIN ST, BOSTON, MA, 02110, US

(21) Appl. No.: 10/332,112

(22) PCT Filed: Jul. 3, 2001
(86) PCT No.: PCT/AU01/00795

(30) Foreign Application Priority Data
Jul. 3, 2000 (AU) PQ 8541

(51) Int. Cl. C12Q 1/24; C12Q 1/04

(52) U.S. Cl. 435/30; 424/164.1; 424/234.1; 424/93.4; 435/34

(57) ABSTRACT

The present invention relates to methods for monitoring treatment of Helicobacter infection and in particular to methods for monitoring eradication of Helicobacter pylori infection using immunoglobulin G2 (IgG2). The invention also relates to methods for predicting the likelihood of successful eradication of Helicobacter infection in a subject to be treated or being treated for the infection and in particular, to methods for predicting the likelihood of successful eradication including determining the levels of interleukin, interferon-γ and IgG in the subject to be, or being treated.

### TECHNICAL FIELD

The present invention relates to methods for monitoring treatment of Helicobacter infection and in particular to methods for monitoring eradication of Helicobacter pylori infection using immunoglobulin G2 (IgG2). The invention also relates to methods for predicting the likelihood of successful eradication of Helicobacter infection in a subject to be treated or being treated for the infection and in particular, to methods for predicting the likelihood of successful eradication including determining the levels of interleukin-4, interferon-γ and IgG in the subject to be, or being treated.

### BACKGROUND ART

Any discussion of the prior art throughout the specification should in no way be considered as an admission that such prior art is widely known or forms part of common general knowledge in the field.

Helicobacter pylori infection is now recognised as an essential pre-requisite for the development of gastric cancer. About 30% of the population become infected with this bacterium and commonly present with chronic gastritis. This may be complicated by gastric or duodenal ulceration, or may present as non-ulcer dyspepsia A sizeable number of carriers are asymptomatic. However, in a small number of patients with H. pylori, their condition evolves through stages (including epithelial cell metaplasia and dysplasia) into neoplasia.

Current Management Practice of H. pylori Infection

Eradication of infection with antibiotics induces an 80-90% cure rate of peptic ulceration. A widely accepted treatment paradigm is based on detection of infection using antibody assays, followed by combination antibiotic therapy without prior endoscopic diagnosis. Endoscopy, before eradication therapy is generally accepted when ‘danger’ symptoms (eg, severe pain, bleeding) occur, or a significant risk of gastric cancer is present However, endoscopy is a procedure which is associated with its own risks and is to be avoided if possible.

H. pylori initiates an IgG antibody response in saliva as well as serum. The serum IgG antibody is the basis of non-invasive diagnosis. Eradication of infection is followed by a very slow fall in serum antibody levels. There has been a study which suggests that IgG antibody levels at 6 months may be of value in assessing successful eradication. Saliva levels of IgG antibody however fall much quicker following eradication, with levels at 6 weeks regularly less than 80% of those prior to antibiotic therapy.

The concept that saliva IgG antibody levels may predict successful eradication, while attractive, proved not to be a practical proposition for monitoring of progress of treatment or eradication of Helicobacter because total IgG antibody levels were unstable to the extent that a viable test in clinical circumstances proved unreliable. At present, no non-invasive stable test exists which would allow successful monitoring of treatment designed to eradicate Helicobacter infection.

Further, in addition to monitoring eradication of H. pylori in individuals treated, it would be desirable to have a test which could be used prior to, or during treatment to determine the likelihood of successful eradication of H. pylori.

It is an object of the present invention to overcome or ameliorate at least one of the disadvantages of the prior art, or to provide a useful alternative.

### SUMMARY OF THE INVENTION

According to a first aspect there is provided a method of monitoring eradication of Helicobacter infection in a subject treated for the infection, including:

a) determination of IgG2 anti-H. pylori antibody level in a saliva sample;

b) comparison of the IgG2 anti-H. pylori antibody level with a predetermined control IgG2 anti-H. pylori antibody level, wherein a reduction in the level of IgG2 anti-H. pylori antibody in the saliva sample compared to the control indicates eradication of Helicobacter.

According to a second aspect there is provided a method of monitoring efficacy of treatment of Helicobacter infection in a subject treated for the infection, including:

a) determination of IgG2 anti-H. pylori antibody level in a saliva sample;

b) comparison of the IgG2 anti-H. pylori antibody level with a predetermined control IgG2 anti-H. pylori antibody level, wherein a reduction in the level of IgG2 anti-H. pylori antibody in the saliva sample compared to the control indicates efficacious treatment of Helicobacter.

According to a third aspect there is provided a method of monitoring relapse or reinfection with Helicobacter in a subject treated for infection with Helicobacter, including:

a) determination of IgG2 anti-H. pylori antibody level in a saliva sample;

b) comparison of the IgG2 anti-H. pylori antibody level with a predetermined control IgG2 anti-H. pylori antibody level, wherein an increase in the level of IgG2 anti-H. pylori antibody in the saliva sample compared to the control indicates relapse or reinfection with Helicobacter.

According to a fourth aspect there is provided a method of detecting unresponsiveness of a subject to treatment of Helicobacter infection, including:

a) determination of IgG2 anti-H. pylori antibody level in a saliva sample;

b) comparison of the IgG2 anti-H. pylori antibody level with a predetermined control IgG2 anti-H. pylori antibody level, wherein lack of change in the level of IgG2 anti-H. pylori antibody in the saliva sample compared to the control indicates lack of response to treatment.

According to a sixth aspect there is provided a kit for monitoring treatment of Helicobacter infection, including,

a) Helicobacter antigen

b) reagent for determining IgG2 subclass antibody.

Preferably, the IgG2 anti-H. pylori antibody is detected by a near-subject assay. The assay may, however, also be a laboratory-based test. Preferably, the assay is an antibody assay although it will be understood that other known methods of measurement can also be effectively used. Most preferably, the assay is an immunoassay such as ELISA, RIA or a similar assay format.

Control levels of IgG2 anti-H. pylori antibody can be established in samples of saliva obtained from normal individuals, ie. those not having an established H. pylori infection. It is preferred however that control levels of IgG2 be determined in subject's own saliva prior to commencement of treatment for infection or, if monitoring relapse or reinfection, the levels of salivary IgG2 following successful eradication of Helicobacter.

According to a seventh aspect, the present invention provides a method of predicting the likelihood of successful eradication of Helicobacter infection in a subject to be treated or being treated for the infection, including:

(i) determination of IL-4 level in a sample from the subject;

(ii) comparison of the IL-4 level with a predetermined control or standard L-4 level,

(iii) wherein a level of IL-4 in the sample from the subject above the control or standard IL-4 level is predictive of the likelihood of successful eradication and a level of IL-4 below the control or standard IL-4 level is predictive of the likelihood of eradication failure.

Preferably, the sample is a blood sample.

Preferably, the IL-4 is detected by an immunoassay and more preferably, it is determined by ELISA.

The skilled addressee will readily be able to identify a suitable control or standard IL-4 level. For example, the control or standard level of IL-4 may be established from analysis of samples obtained from subjects not infected by H. pylori and/or subjects having successfully eradicated H. pylori and/or subjects infected by H. pylori.

According to an eighth aspect, the present invention provides a method of predicting the likelihood of successful eradication of Helicobacter infection in a subject to be treated or being treated for the infection, including:

(i) determination of interferon-γ (INF-γ) level in a sample from the subject;

(ii) comparison of the INF-γ level with a predetermined control or standard INFIX level,

(iii) wherein a level of INF-γ in the sample from the subject below the control or standard INF-γ level is predictive of the likelihood of successful eradication and a level of IFN-γ above the control or standard level is predictive of the likelihood of eradication failure.

Preferably, the INF-γ level is determined in a blood sample.

Preferably, the INF-γ level is detected by an immunoassay and preferably the assay is ELISA.

The skilled addressee will readily be able to establish a suitable control or standard. For example, the control or standard level of INF-γ may be established from analysis of samples obtained from subjects not infected by H. pylori and/or subjects having successfully eradicated H. pylori and/or subjects infected by H. pylori.

According to a ninth aspect, the present invention provides a method of predicting the likelihood of successful eradication of Helicobacter infection in a subject to be treated or being treated for the infection, including:

(i) determination of immunoglobulin G (IgG) level in a sample from the subject;

(ii) comparison of the IgG level with a predetermined control or standard IgG level,

(iii) wherein a level of IgG in the sample from the subject below the control or standard level is predictive of the likelihood of successful eradication and a level of IgG above the control or standard level is predictive of the likelihood of eradication failure.

Preferably, the IgG level is determined in a serum sample and, more preferably, the sample is a saliva sample.

The skilled addressee will readily be able to establish a suitable control or standard level of IgG. For example, the control or standard level of IgG may be established from analysis of samples obtained from subjects not infected by H. pylori and/or subjects having successfully eradicated H. pylori and/or subjects infected by H. pylori.

According to a tenth aspect, the present invention provides a method of predicting the likelihood of successful eradication of Helicobacter infection in a subject to be treated or being treated for the infection, including:

(i) determination a combination of IL-4 and/or INF-γ and/or IgG levels in a sample from the subject;

(ii) comparison of the IL-4 and/or INF-γ and/or IgG levels with a predetermined control or standard IL-4 and/or INF-γ and/or IgG level respectively,

wherein a level of IL-4 in the sample from the subject above the control or standard level is predictive of the likelihood of successful eradication and a level of IL-4 below the control or standard level is predictive of the likelihood of eradication failure, and

wherein a level of INF-γ in the sample from the subject below the control or standard level is predictive of the likelihood of successful eradication and a level of INF-γ above the control or standard level is predictive of the likelihood of eradication failure, and

wherein a level of IgG in the sample from the subject below the control or standard level is predictive of the likelihood of successful eradication and a level of IgG above the control or standard level is predictive of the likelihood of eradication failure.

### BRIEF DESCRIPTION OF THE FIGURES

FIG. 1 Stability of salivary IgG2 anti-Helicobacter pylori antibody.

FIG. 2 Salivary IgG (panel A) and IgG2 (panel B) anti-H. pylori antibody before and after eradication of H. pylori.

FIG. 3 Salivary IgG (panel A) and IgG2 (panel B) anti-H. pylori antibody in subject with and without H. pylori infection.

FIG. 4 Correlation between IL-4 production in whole blood and gastric tissue cultures. Whole blood cultures or gastric antrum biopsy cultures were incubated for 24 hours at 37° C., after which time the levels of IL-4 were measured by ELISA capture assay. The results shown a correlation between mucosal and whole blood IL-4 (p<0.001).

FIG. 5 Levels of IL-4 in whole blood culture stimulated with H. pylori AGE antigen. Peripheral blood obtained from subjects with or without H. pylori infection, or with eradication failure was added to equal volume of AIM-V culture medium containing graded concentrations of H. pylori AGE antigen as indicated. After 24 hours of culture, levels of IL-4 were measured by ELISA capture assay. Results shown are the mean±standard error of the mean. *:p<0.05: compared with H. pylori-eradicated subjects; ¶: p<0.01 and p<0.05 compared with the values from subjects with H. pylori-eradicated and H. pylori-positive, respectively.

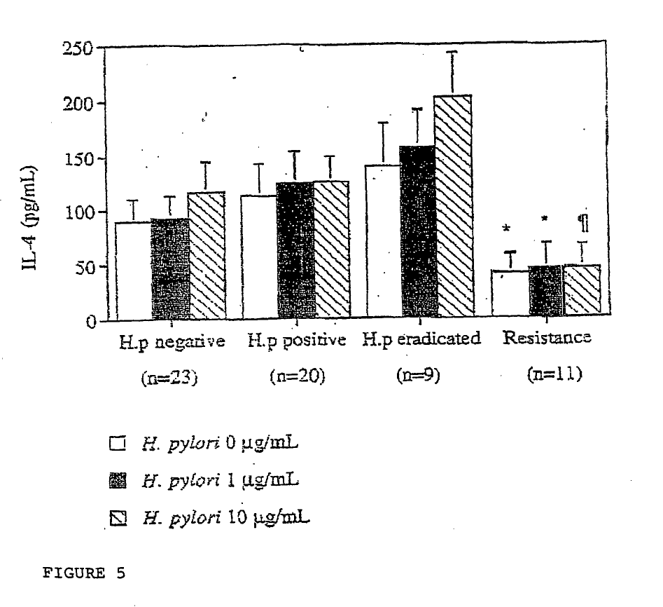

FIG. 6 INF-γ production in response to H. pylori acid-glycine extract stimulation in whole blood. Peripheral blood was collected from individual subject and cultured in the presence of graded concentration of H. pylori AGE antigen for 24 hours. Culture supernatants were collected and assayed for IFN-γ by ELISA. Results shown were mean±standard error of the mean. NS: Not Significant.

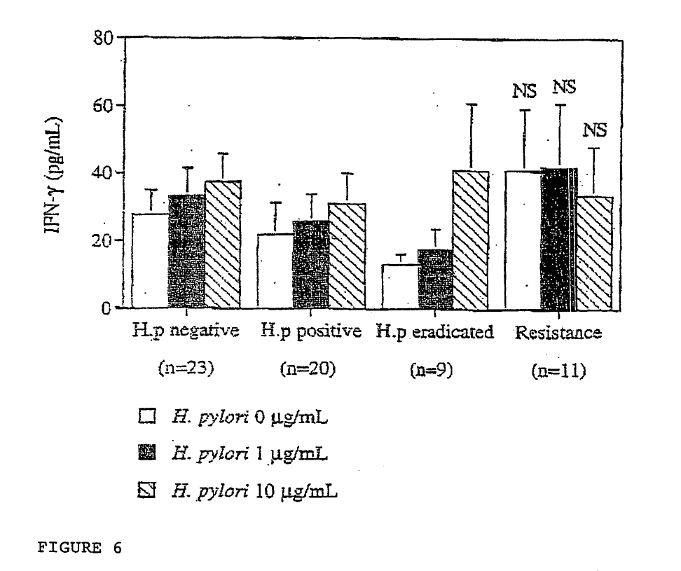

FIG. 7 Levels of specific H. pylori IgG antibody in serum and saliva. Serum and saliva samples were collected from individual subjects. Levels of specific H. pylori IgG were measured by ELISA. Results shown were mean±standard error of the mean. *: p<0.05 compared with mean from H. pylori-positive group; NS: Not Significant.

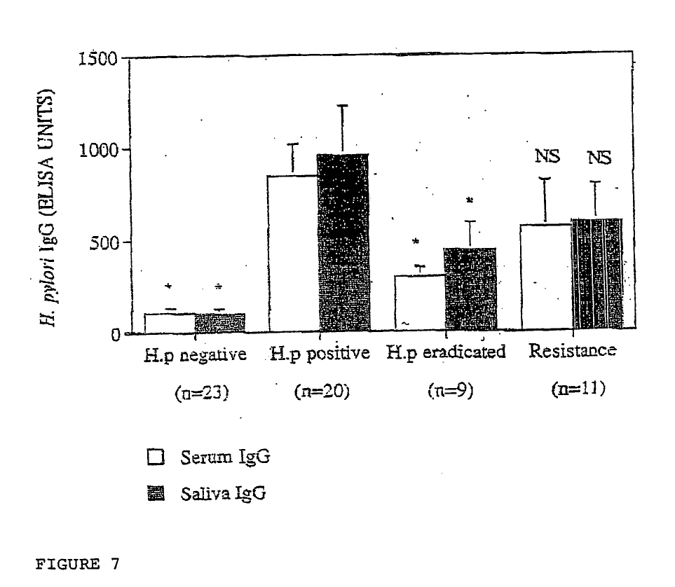

### DESCRIPTION OF THE PREFERRED EMBODIMENTS

It has surprisingly been found that salivary IgG2 anti-H. pylori antibody is stable and allows a reliable test to be developed for monitoring progress of treatment and/or eradication of Helicobacter pylori infection in a subject undergoing treatment.

It was previously known that IgG anti-H. pylori antibody levels in blood and gastric mucosa can be used as an indicator of H. pylori status. There has been an attempt to use IgG anti-H. pylori antibody in saliva for a similar purpose but it proved to be unstable in such a sample. From the following examples it will be understood that while IgG anti-H. pylori may be useful as a general indicator of H. pylori status, it is the measurement of the IgG2 subclass anti-H. pylori antibody which allows a stable treatment monitoring test to be developed.

It has further surprisingly been found that IL-4 levels can be used as a predictor of successful eradication of H. pylori. It is envisaged that an IL-4 test could be used prior to, or during the treatment of H. pylori infection in order to predict the likelihood of eradication.

Techniques for measurement of antibodies and IL-4 in human samples are well-known in the art and protocols and reagents are readily available. Examples of some of the techniques used are indicated below as an illustration of how some measurements may be performed.

Unless indicated otherwise, standard techniques which can be ascertained from standard texts and laboratory manuals may be employed.

The invention will now be described in more detail with reference to non-limiting examples.

### EXAMPLES

### Example 1

### Determination of Antibody Levels in Saliva Samples

Sample Collection

Saliva samples were collected from 4 patients infected with H. pylori who were treated with eradication triple therapy comprised of amoxycillin, omeprazole and clarithromycin for seven days. Samples were taken before treatment and after 10 days of eradication therapy.

H. pylori Antigen Preparation

H. pylori NCTC 11637 strain was used for H pylori antigen preparation according to modified methods described by Goodwin (#208). Protein concentration in the extract was measured using a bio-rad kit (Bio-rad laboratories Australia). Aliquots were stored at −70° C.

Antibody Detection by ELISA

For saliva anti-H. pylori antibody detection, wells of a 96-well flat-bottomed microtiter Polysorb plate (Nunc, Denmark) were coated with 7 μg/mL of H. pylori antigen at 4° C. overnight. After washing and blocking the plates with 5% skim milk (Diploma, Australia) in PBS-Tween 20, saliva samples at 1:2 dilution with 2% PEG 6000 were added to individual wells in triplicate. After incubation, the wells were washed and horseradish peroxidase conjugated-sheep anti-IgG or anti-IgG2 (Silenus, Australia) at 1:2000 dilution was added to each well. Following a further incubation, the plates were washed and then tetramethyl benzidine (TMB) substrate (Sigma, USA) was added to each well. The reaction was stopped using 1 mol/L H₂SO₄ and the absorbance was read at 450 nm in an ELISA plate reader (BioRad 450, Japan). The results were expressed as ELISA INDEX being the mean OD₄₅₀ of a given saliva sample divided by the mean OD₄₅₀ of the calibrating sample. Positive and negative quality control samples were included in each run to control for intra- and inter-assay variation.

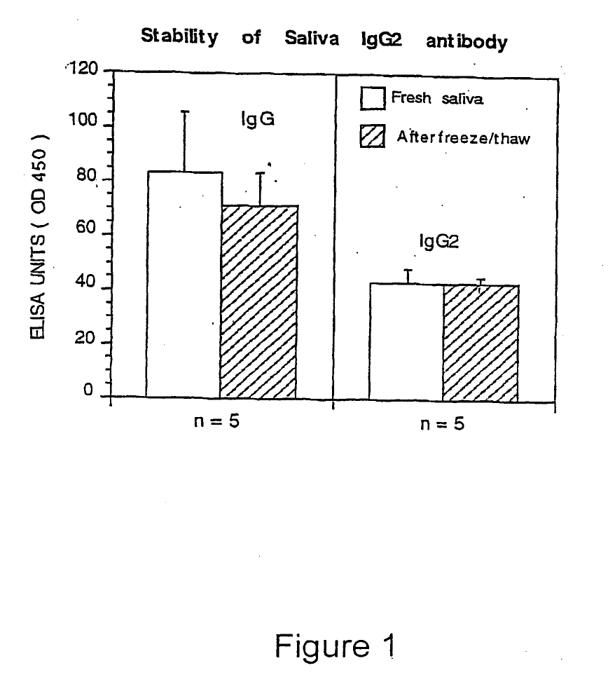

Saliva samples were obtained from 5 subjects infected with H. pylori. The samples were tested for IgG2 and total IgG anti-H. pylori antibody by the ELISA assay either fresh or after storage for up to 12 months. The results show that IgG2 antibody levels were more stable than IgG antibody levels (FIG. 1). Hence, IgG2 antibody is a reliable and a sensitive indicator of infection status due to its stability during storage and assay.

### Example 2

### Anti-H. pylori Antibody Levels in Saliva from Patients Undergoing Eradication therapy.

Saliva samples from subjects undergoing antibiotic eradication therapy were tested for anti-H. pylori antibody using the immunoassay method described in Example 1.

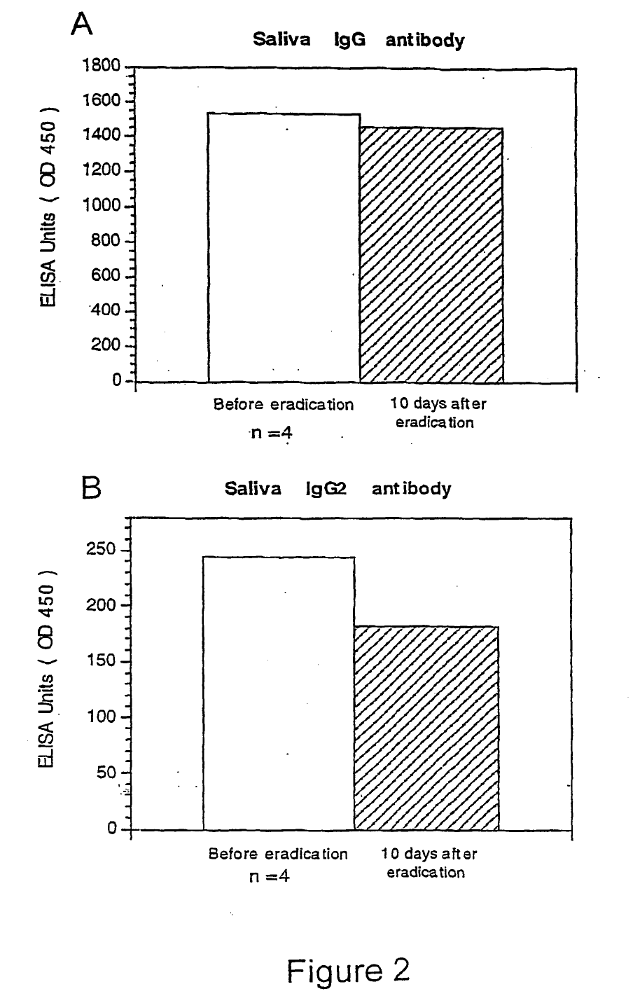

IgG and IgG2 antibody was measured before and after treatment with antibiotics. Ten days after treatment IgG2 antibody levels fell quicker than total IgG antibody levels (FIGS. 2A and 2B).

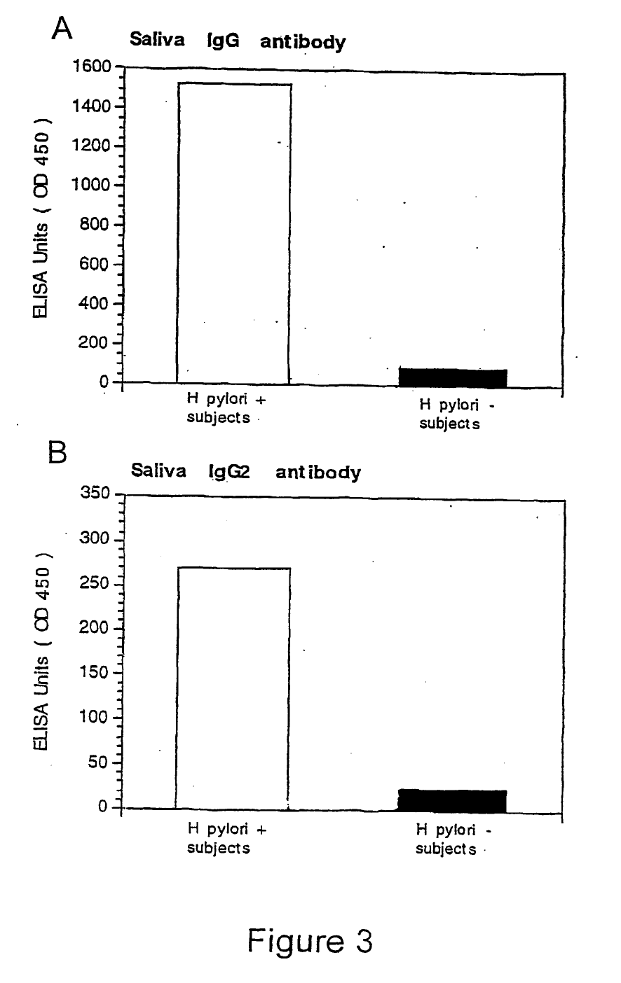

In a separate study it was shown that saliva from subjects with H. pylori infection have markedly elevated levels of IgG2 (FIG. 3A) when compared to subjects without infection (FIG. 3B). Subjects who failed to ultimately eradicate the infection did not demonstrate a significant drop in the level of IgG2 anti-H. pylori antibody.

### Example 3

### Interleukin-4/IFN-γ and IgG Studies

Subjects

Fifty-two subjects referred for investigation of dyspepsia, and 11 subjects with persistent H. pylori infection following one or more courses of antibiotics, were recruited for this study. Subjects with dyspepsia had not taken antibiotics within three months of the study. The study was approved by the Ethics Committee of the Centre for Digestive Diseases, Sydney, Australia Informed consent was obtained from all patients. Multiple biopsy specimens were obtained during upper gastrointestinal endoscopy from the antrum and the body of the stomach to be used for tissue culture, histology and a urease test (CLO test, Delta West, WA, Australia). Blood samples were incubated at 37° C. within 2 hours of collection. Serum was stored at −70° C. for H. pylori specific antibody.

Saliva Sample Collection

Saliva samples were collected before the endoscopy procedure. Samples were centrifuged at 1000×g for 10 minutes at 4° C., and aliquots were stored at −70° C.

Biopsy Culture

Gastric biopsy tissues were weighted and cultured at a ratio of 50 μL serum-free AIM-V medium (Life Technology, Australia) per mg tissue (wet weight) for 24 hours. The culture supernatants were collected and centrifuged. Aliquots were stored at −70° C. until assay.

H. pylori Antigen Preparation

H. pylori antigens from the NCTC 11637 strain were prepared by acid-glycine extraction (AGE) according to the method described by Goldwin et al (J Infect Dis 1987; 155:488-94). H. pylori AGE was used for cell culture and specific antibody measurement.

ELISA Capture Assay for IL-4 in Whole Blood Culture

Cytokine levels in whole blood culture were measured following the method described previously (Ren et al, Helicobacter 2000; 5:135-41). Briefly, 150 AL of heparinized whole blood was added in triplicate to wells of a 96-well microtitre flat-bottomed plate pre-coated with mouse polyclonal anti-human IL-4 antibody (Endogen, MA, USA). An equal volume of AIM-V medium containing H. pylori AGE at either 0, 1 or 10 μg/mL was also loaded to wells. The cultures were incubated at 37° C. with 5% CO₂ for 24 hours, after which time supernatants were collected for interferon-γ (IFN-γ) assay. The amount of ‘captured’ IL-4 was measured by ELISA as following. Briefly, after washing the plates, biotinylated mouse monoclonal anti-human IL-4 antibody (Endogen, MA, USA) was added (0.5 μg/mL) to wells and incubated for 90 minutes at room temperature. The plates were then washed and incubated for a further 30 minutes at room temperature with streptavidin-conjugated horseradish peroxidase (Selinus, Australia) at a 1:400 dilution. The plates were thoroughly washed with washing buffer and finally incubated for 10 minutes at room temperature with 3,3′-5,5′ tetramethyl benzidine (TMB, Sigma-Aldrich, USA) substrate. The reaction was stopped using 1 mol/L H₂SO₄ and optical density at 450 nm (OD 450 nm) was measured in an ELISA plate reader (Bio-Rad 450, Japan). Standard IL-4 (Endogen, MA, USA) was applied for each plate to control plate to plate variation. The limits of sensitivity for IL-4 was 9.4 μg/mL. The amount of IL-4 in samples was determined using a Softmax program (Version 2.3 FPU, USA).

INF-γ ELISA Assay

Wells of a 96-well flat-bottomed microtitre plate (Nunc, Denmark) were coated with mouse anti-human IFN-γ monoclonal antibody (Endogen, MA, USA) at 2 μg/1 mL overnight at 4° C. After washing and blocking, supernatants from whole blood culture or IFN-γ standards (Endogen, MA, USA) were added in duplicate, and incubated for 90 minutes. The plates were washed and biotinylated mouse monoclonal anti-human IFN-?γ antibody (Endogen, MA, USA) was added (0.25 μg/mL). After 90 minutes incubation, the wells were washed and streptavidin-conjugated horse-radish peroxidase (Selinus, Australia) was applied at a 1:2000 dilution. The plates were washed and TMB chromagen (Sigma-Aldrich, USA) was added to each well. The absorbance was read at 450 nm in an ELISA plate reader (Bio-Rad 450, Japan). The limits of sensitivity for INF-γ was 9.4 μg/mL. The amount of IFN-γ in samples was determined using a Softmax program (Version 2.3 FPU, USA).

Detection of H. pylori Antibody

Wells of a 96-well flat-bottomed microtitre plate were coated with H. pylori AGE at 5 μg/mL at 4° C. overnight. After washing and blocking, serum samples at 1:3000 dilution and saliva sample at 1:4 dilution were added to wells in triplicate. Horse-radish peroxidase conjugated-sheep anti-IgG (Selinus, Australia) was applied at 1:2000 dilution. Tetramethyl Benzidine (TMB) substrate (Sigma-Aldrich, USA) was used for colour development. The absorbance was read at 450 nm in an ELISA plate reader (Bio-Rad, 450, Japan). The results were expressed as ELISA Units against a reference standard of pooled positive sera. Intra- and inter-assay variation was less than 10%.

Statistical Analysis

Data were expressed as mean±standard error (SE). Correlation Z test was used to test for a correlation between mucosal and blood cytokine production. Differences of means among patient groups were analysed by ANOVA. All statistical analysis were performed by using a StatView 4.5 software program (Abacus Concepts, California, USA). Significant difference was considered when p value was less than 0.05.

Results

Subjects were divided into four groups according to H. pylori infection status and results of antibiotic treatment. There were 23 H. pylori-negative subjects; 20 H. pylori-positive subjects; 9 subjects with successful H. pylori eradication confirmed by histology or C¹⁴ breath test at 6-8 weeks after eradication therapy; and 11 subjects with H. pylori resistance following antibiotic therapy. Details of diagnosis and therapeutic regimens in subjects with eradication failure are shown in Table 1.

Comparison of Blood and Mucosal IL-4 Response

To determine whether there is a correlation between blood and mucosal cytokine responses to H. pylori infection, levels of IL-4 production in whole blood cultures stimulated or unstimulated with H pylori antigens, were compared with levels in gastric mucosa cultures (FIG. 1) (data from antigen stimulated cultures not shown). The results from H pylori positive (n=6) and negative subjects (n=11) and subjects with failed eradication (n=8) showed that IL-4 production in whole blood cultures (stimulated or unstimulated) correlated with that in gastric mucosa (r²=0.549, p<0.001).

IL-4 and IFN-γ Production in Whole Blood Culture

Significantly lower levels of IL-4 were detected in whole blood stimulated or unstimulated with H. pylori AGE from subjects with eradication failure compared with subjects in whom H. pylori was successfully eradicated (p<0.05, 0 and 1.0 μg/mL H. pylori AGE; p<0.01, 10 μg/mL H. pylori AGE) or in subjects with untreated infection (p<0.05, 10 μg/mL H. pylori AGE) (FIG. 2). IL-4 levels were similar in non-infected and infected subjects, and were not significantly different when compared to subjects with successful eradication (though there was a trend towards increased levels following eradication). Although there was no statistically significant difference in the levels of IFN-γ between the different groups, lower levels were detected in subjects with successful H. pylori eradication (FIG. 3). Low levels of IL-4 secretion were seen in most subjects with ongoing infection with resistant H. pylori, irrespective of the number of courses of therapy (Table 2).

Anti-H. pylori IgG Levels in Serum and Saliva

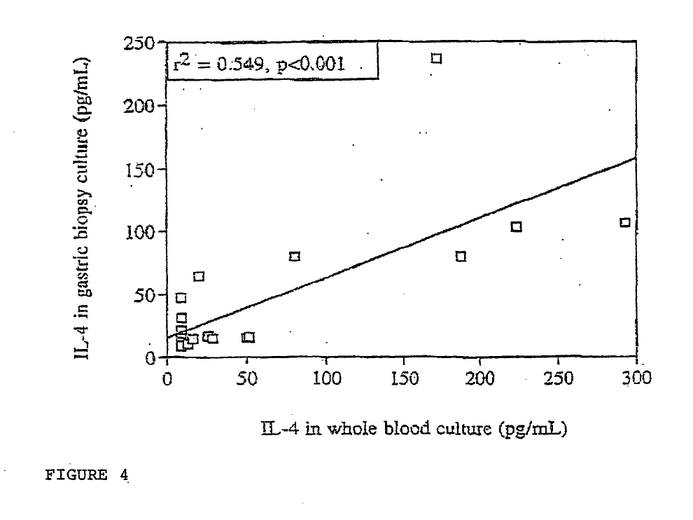

Both serum and saliva IgG antibody levels were significantly lower in non-infected subjects (p<0.05) and in subjects at 6-8 weeks after eradication therapy (p<0.05) than in subjects who were positive for H. pylori. For both saliva and serum antibody, a trend towards lower levels of antibody in those failing to eradicate infection was seen, but this was short of statistical significance (FIG. 4).

TABLE 1
Clinical Characterisation of Subjects with Failed Antibiotic Therapy

| | | | | Number of | |
|---|---|---|---|---|---|
| | Age | | | Antibiotic | Duration |
| No. | (years) | Diagnosis | Treatment Regimens Used | Courses | (months) |
| 1 | 40 | Hp-induced gastritis | metronidazole/amoxicillin/bismuth/ranitidine HCl | 1 | 24 |
| | | | clarithromycin/metronidazole/lansoprazole/amoxicillin | 2 | |
| 2 | 58 | Hp-induced gastritis | Losec HP7 | 1 | 12 |
| 3 | 55 | Oesophagitis and Hp-induced gastritis | Klacid HP7 | 1 | >3 yrs |
| | | | Helidac/ranitidine HCl | 1 | |
| | | | lansoprazole | 1 | |
| 4 | 47 | Hp-induced gastritis | Losec HP7 | 2 | 20 |
| 5 | 37 | Hp-induced gastritis | metronidazole | 1 | 5 |
| | | | Losec HP7 | 3 | |
| 6 | 45 | Hp-induced gastritis | Losec HP7/ranitidine HCl | 3 | 28 |
| 7 | 27 | Hp-induced gastritis | Losec HP7 | 3 | 6 |
| | | | clarithromycin/tetracycline/metronidazole/lansoprazole | 1 | |
| 8 | 33 | Hp-induced gastritis and | Helidac | 2 | >3 yrs |
| | | duodenal ulcer disease | | | |
| 9 | 26 | Hp-induced gastritis | Losec HP7 | 2 | 10 |
| 10 | 47 | Hp-induced gastritis | Losec HP7 | 3 | >3 yrs |
| 11 | 73 | Oesophagitis, Hp-induced gastritis and | Losec HP7 | 3 | >3 yrs |
| | | duodenal ulcer disease | | | |

Hp = Helicobacter pylori; Helidac = bismuth/metronidazole/tetracycline; Klacid HP7 = omeprazole/amoxicillin/clarithromycin; Losec HP7 = omeprazole/amoxicillin/clarithromycin

TABLE 2
IL-4 and H. pylori Antibody IgG in Subjects with Failure Eradication

| | | IL-4 levels | | | H. pylori | |
|---|---|---|---|---|---|---|
| | | (pg/mL)* | | | Antibody IgG* | |
| Times of | No. | H. pylori antigen | H. pylori antigen | H. pylori antigen | Serum | Saliva |
| failure | Subjects | (0 μg/mL) | (1 μg/mL) | (10 μg/mL) | (ELISA Unit) | (ELISA Unit) |
| One | 1 | 20.76 | 28.21 | 44.20 | 214 | 116.3 |
| Two | 3 | 40.49 ± 29.36 | 54.07 ± 43.14 | 65.22 ± 45.86 | 224 ± 101.58 | 1000.2 ± 866.5 |
| Three | 5 | 45.16 ± 36.16 | 53.34 ± 44.34 | 55.63 ± 44.19 | 410.95 ± 167.29 | 418.9 ± 151.96 |
| Four | 2 | 18.82 ± 9.82 | 22.56 ± 13.58 | 12.60 ± 3.6 | 1453.6 ± 1244.4 | 523.7 ± 235.3 |

*Standard error of mean (SEM).

The skilled addressee will understand that, in light of the above, IL-4, INF-γ and IgG can be used to predict the likelihood of successful eradication of Helicobacter infection before or during treatment of the infection. As a corollary, it will be clear that the method can also be used to identify subjects unlikely to respond to treatment for Helicobacter infection.

Although the invention has been described with reference to specific examples, it will be appreciated by those skilled in the art that the invention may be embodied in many other forms without departing from the spirit or intent of the inventive concept.

The claims defining the invention are as follows:

1. A method of monitoring eradication of Helicobacter infection in a subject treated for the infection, including:
i) determination of IgG2 anti-H. pylori antibody level in a saliva sample;
ii) comparison of the IgG2 anti-H. pylori antibody level with a predetermined control IgG2 anti-H. pylori antibody level, wherein a reduction in the level of IgG2 anti-H. pylori antibody in the saliva sample compared to the control indicates eradication of Helicobacter.

2. A method of monitoring efficacy of treatment of Helicobacter infection in a subject treated for the infection, including:
i) determination of IgG2 anti-H. pylori antibody level in a saliva sample;
ii) comparison of the IgG2 anti-H. pylori antibody level with a predetermined control IgG2 anti-H. pylori antibody level, wherein a reduction in the level of IgG2 anti-H. pylori antibody in the saliva sample compared to the control indicates efficacious treatment of Helicobacter.

3. A method of monitoring relapse or reinfection with Helicobacter in a subject treated for infection with Helicobacter, including:
i) determination of IgG2 anti-H. pylori antibody level in a saliva sample;
ii) comparison of the IgG2 anti-H. pylori antibody level with a predetermined control IgG2 anti-H. pylori antibody level, wherein an increase in the level of IgG2 anti-H. pylori antibody in the saliva sample compared to the control indicates relapse or reinfection with Helicobacter.

4. A method of detecting unresponsiveness of a subject to treatment of Helicobacter infection, including:
(i) determination of IgG2 anti-H. pylori antibody level in a saliva sample;
(ii) comparison of the IgG2 anti-H. pylori antibody level with a predetermined control IgG2 anti-H. pylori antibody level, wherein lack of change in the level of IgG2 anti-H. pylori antibody in the saliva sample compared to the control indicates lack of response to treatment.

5. A method according to any one of claims 1 to 4, wherein the IgG2 anti-H. pylori antibody is detected by an immunoassay.

6. A method according to claim 5, wherein the assay is ELISA.

7. A method according to any one of claims 1 to 6, wherein the control levels of IgG2 anti-H. pylori antibody is established in samples of saliva obtained from subjects not infected by H. pylori.

8. A method according to any one of claims 1 to 6, wherein the control levels of IgG2 anti-H. pylori antibody are determined in subject's own saliva sample.

9. A kit for monitoring treatment of Helicobacter infection, including,
(i) Helicobacter antigen
(ii) reagent for determining IgG2 subclass antibody.

10. A method of predicting the likelihood of successful eradication of Helicobacter infection in a subject to be treated or being treated for the infection, including:
(i) determination of IL-4 level in a sample from the subject;
(ii) comparison of the IL-4 level with a predetermined control or standard IL-4 level,
(iii) wherein a level of IL-4 in the sample from the subject above the control or standard IL-4 level is predictive of the likelihood of successful eradication and a level of IL-4 below the control or standard IL-4 level is predictive of the likelihood of eradication failure.

11. A method according to claim 10 wherein the sample is a blood sample.

12. A method according to claim 10 or claim 11, wherein the IL-4 is detected by an immunoassay.

13. A method according to claim 12, wherein the assay is ELISA.

14. A method according to any one of claims 10 to 13, wherein the control or standard level of IL-4 is established from analysis of samples obtained from subjects not infected by H. pylori and/or subjects having successfully eradicated H. pylori and/or subjects infected by H. pylori.

15. A method of predicting the likelihood of successful eradication of Helicobacter infection in a subject to be treated or being treated for the infection, including:
(i) determination of interferon-γ (INF-γ) level in a sample from the subject;
(ii) comparison of the INF-γ level with a predetermined control or standard INF-γ level,
(iii) wherein a level of INF-γ in the sample from the subject below the control or standard INF-γ level is predictive of the likelihood of successful eradication and a level of INF-γ above the control or standard level is predictive of the likelihood of eradication failure.

16. A method according to claim 15 wherein the sample is a blood sample.

17. A method according to claim 15 or claim 16, wherein the IFN-γ level is detected by an immunoassay.

18. A method according to claim 17, wherein the assay is ELISA.

19. A method according to any one of claims 15 to 18, wherein the control or standard level of IFN-γ is established from analysis of samples obtained from subjects not infected by H. pylorii and/or subjects having successfully eradicated H. pylori. and/or subjects infected by H. pylori.

20. A method of predicting the likelihood of successful eradication of Helicobacter infection in a subject to be treated or being treated for the infection, including:
(i) determination of immunoglobulin G (IgG) level in a sample from the subject;
(ii) comparison of the IgG level with a predetermined control or standard IgG level,
(iii) wherein a level of IgG in the sample from the subject below the control or standard level is predictive of the likelihood of successful eradication and a level of IgG above the control or standard level is predictive of the likelihood of eradication failure.

21. A method according to claim 20 wherein the sample is a serum sample.

22. A method according to claim 20 wherein the sample is a saliva sample.

23. A method according to any one of claims 20 to 22, wherein the control or standard level of IgG is established from analysis of samples obtained from subjects not infected by H. pylori and/or subjects having successfully eradicated H. pylori and/or subjects infected by H. pylori.

24. A method of predicting the likelihood of successful eradication of Helicobacter infection in a subject to be treated or being treated for the infection, including:
(i) determination a combination of IL-4 and/or INF-γ and/or IgG levels in a sample from the subject;
(ii) comparison of the IL-4 and/or INF-γ and/or IgG levels with a predetermined control or standard L-4 and/or IF-γ and/or IgG level respectively, wherein a level of IL-4 in the sample from the subject above the control or standard level is predictive of the likelihood of successful eradication and a level of IL-4 below the control or standard level is predictive of the likelihood of eradication failure, and
wherein a level of INF-γ in the sample from the subject below the control or standard level is predictive of the likelihood of successful eradication and a level of IFN-γ above the control or standard level is predictive of the likelihood of eradication failure, and
wherein a level of IgG in the sample from the subject below the control or standard level is predictive of the likelihood of successful eradication and a level of IgG above the control or standard level is predictive of the likelihood of eradication failure.
